(19) United States
(12) Patent Application Publication
(10) Pub. No.: US 2018/0280287 A1
(43) Pub. Date: Oct. 4, 2018

(54) METHOD AND COMPOSITION FOR A SACHA INCHI PERENNIAL PLANT EXTRACT BASED HEALTH AND BEAUTY AID

(71) Applicant: TRI-K Industries, Inc., Denville, NJ (US)

(72) Inventors: Mihaela Gorcea, Woodland Park, NJ (US); Dmitri Ptchelintsev, Jersey City, NJ (US)

(21) Appl. No.: 15/913,025

(22) Filed: Mar. 6, 2018

Related U.S. Application Data
(60) Provisional application No. 62/481,423, filed on Apr. 4, 2017.

(51) Int. Cl. A61K 8/9783; A61K 8/81; A61K 8/41; A61K 8/37; A61K 8/34; A61K 8/02; A61Q 19/08

(52) U.S. Cl. CPC A61K 8/9783; A61K 8/8147; A61K 8/41; A61K 8/37; A61K 2800/805; A61K 8/342; A61K 8/0212; A61Q 19/08; A61K 8/345

(57) ABSTRACT

A method and composition for non-invasive treatment of skin aging conditions utilizes a topically applied composition that includes a sustainable non GMO water based extract of peptide Sacha Inchi (hydrolyzed Plukenetia Volubilis Seed Extract). The composition utilizes Sacha Inchi to clinically and non-invasively reduce after only 28 days skin sagginess and facial volume contour. Reshaping and resculpting facial skin and reducing dermal non-echogenic surfaces are also accomplished. Increased echogenic surfaces, increased dermal connective tissue and dermal density by restructuring, re-densifying and revitalizing dermis for healthier skin is furthered. The composition was clinically proven to reduce after only 21 days of treatment fine lines, deep wrinkles and facial skin roughness, for a smoother, healthier, younger and less wrinkled skin. The composition was also clinically proven to reduce sebum production, minimize pore size, mattifying skin appearance by sebum regulation and smoothing the appearance of skin after only 21 days of treatment.

### CROSS-REFERENCE TO RELATED APPLICATION

The present application claims the benefit of the filing date of U.S. Provisional Patent Application No. 62/481,423, filed Apr. 4, 2017, the disclosure of which is hereby incorporated herein by reference.

### FIELD OF USE

The present disclosure relates to the fields of health and beauty aids. In particular, the present disclosure relates to the field of non-invasive treatment of skin aging using a Sacha Inchi perennial plant extract health and beauty aid.

### BACKGROUND

It is known that chronological aging, photoaging, genetics, diet, stress, weight fluctuations or exposure to environmental toxins can cause a gradual loss of connective tissue and dermal matrix components such as collagen, elastin and glycosaminoglycans. Over time, this leads to reduction in skin thickness, loss of dermis density, decrease of orderly deposition and properly alignment of collagen and elastin fibers within the dermal matrix, and ultimately to skin sagginess, laxity, lack of firmness and appearance of fine lines and deep wrinkles and overall aged skin.

Traditionally, such manifestations of skin aging have been addressed with invasive medical and dermatological methods such as cosmetic surgeries, plastic surgeries, facial lifts, chemical acid peels. The invasive methods can lead to skin irritation, inflammation, itching, edema, redness, skin discoloration, discomfort and pain. Traditionally, skin aging conditions have been treated by topical applications of composition containing retinol, retinoic acid and their analogues. It well known that retinoids and their derivatives have several dramatic side effects such as teratogenicity, redness, inflammation, irritation, skin dryness, flaking, peeling, sensitivity to sunlight, chemical instability in topical formulations. Other cosmetic treatments of treating aging skin conditions are topical applications containing hydroxycarboxylic acids, polyhydroxy acids and oxacarboxylic acids (glycolic, lactic, polybionic acids, lactobionic acid) which are known to cause skin stinging sensation, redness, irritation, inflammation, discomfort, decreasing stratum corneum thickness.

Surgical methods are well known in the art for various skin conditions such as saggy skin, droopy skin, skin with lack of firmness, or lack of elasticity and vigor, skin with laxity, fine lined skin, deep wrinkles, and skin with rough texture. Often these severe skin conditions are due to aging, genetic predispositions, changes in metabolism, or other medical conditions. Non-surgical methods for treating these skin conditions have been met with limited success to date.

US 2007/0264221 to Moser et al. discloses a plant extract that contains proteins which when applied to the skin can provide an anti-inflammatory effect, a skin tightening effect, an antiaging effect, and a substantively effect. Moser et al describes Plukenetia volubilis L, also called “Inca peanut” or “Inca Inchi” or “Sacha Inchi” that is a plant native to the high-altitude rain forests of the Andean region of South America. Despite its vernacular name “Inca peanut”, it does not belong to the same botanical family as the true peanut (Arachis hypogea L-family Fabaceae), but to the family Euphorbiaceae. Moser et al describes Plukenatia volubilis “oil” that is mixed with the flour meal by Mayorunas, Chayuhuitas, Campas, Huitotas Shipibas, Yaguas and Baras women to make a cream to revitalize and give youth to the skin. The seed kernels of Plukenetia volubilis are rich in oil (35 to 60%). This oil is characterized by a high content in unsaturated fatty acids, around 37% of linoleic acid and 50% of alpha-linolenic acid with high nutritional value.

However Moser et al. states only little information is available in the state of the art on the contents of Plukenetia kernels, and furthermore Moser et al. fails to provide any combination or compositions that may be useful in treating the above mentioned severe skin conditions. Lastly, Moser et al. composition reports a molecular weight of 30,000 and 60,000 Da (Dalton, where 1 Da=1 g/mol) that is a relatively large molecule.

Furthermore, past attempts to address the need for a non-invasive solution that provides a remedy to the above skin conditions have been met with limited success or failed. For example, like the above compositions, including Plukenatia volubilis oil or Sachi Inchi, are very expensive and are known to cause nausea if ingested. Furthermore, many skin compositions include additives such as animal by-products, gluten, or Genetically Modified Organisms (GMO) that may have long term health consequences.

Thus there still remains a need for a composition and method of use for the promotion of healthier skin without the above shortcomings and drawbacks. Furthermore there also remains a need in the art for a composition and method of use that avoids the use of substances such as genetically modified organisms (GMO).

### SUMMARY OF THE INVENTION

The present invention solves the problems of current state of the art and provides many more benefits. Discloses is a method and composition for a topically applied formulation containing sustainable non GMO natural peptide Sacha Inchi (Hydrolyzed Plukenetia Volubilis Seed Extract) that specifically treats non-invasively these skin conditions that are difficult to treat such as: saggy skin, droopy skin, skin with lack of firmness, elasticity and vigor, skin with laxity, skin with fine lines, deep wrinkles and rougher texture.

The present invention utilizes a non-oil based substance. Specifically, a water based extract of Sacha Inchi is used in the present invention to improve skin conditions that are difficult to treat using non-invasive methods and were not investigated with previous oil based Sacha Inchi substances. For example, water based extract of Sacha Inchi of the present invention has a comparably lower average molecular weight below or equal to about, i.e., 2,500 Da, while compositions like in the above reference Moser et al. reports have a much higher molecular weight of 30,000 and 60,000 Da. The two compositions, water based extract of Sacha Inchi and previously reported Moser et al. composition, are different chemically and have different molecular weights.

Objects of the invention include, but are not limited to, the following subject matters: preparing a new active composition prepared from the seeds of Plukenetia Volubilis with a unique biotechnological process; topical compositions containing the above-mentioned active material; and developing a new way of treating skin conditions comprising topically applying compositions described in the above objects.

To our knowledge the present invention is a unique water based extract of Sacha Inchi prepared from the seeds of Plukenetia Volubilis which has a different chemical composition than previous compositions and contain unique amino acids and small peptides composition due to a unique botanical source compared to the oil soluble Sacha Inchi compositions known in the past.

Specifically, this invention relates to a new composition containing hydrolyzed Plukenetia Volubilis (Sacha Inchi) Seed Extract and a novel method of treating ageing skin using such composition that specifically improves non-invasively the symptoms of aging skin which are difficult to treat such as: saggy skin, droopy skin, skin with lack of firmness and elasticity, skin with laxity, skin with fine lines, deep wrinkles and rougher skin texture. More particularly, the invention relates to the novel topical composition containing hydrolyzed Plukenetia Volubilis (Sacha Inchi) seed extract clinically proven to reduce skin sagginess, reshape and re-sculpt the facial contour.

The present invention water based extract of Sacha Inchi provides many advantages versus oil based compositions. These advantages include but are not limited to, no sticky or greasy residue after application on skin, lighter texture compared to an oil based formulation, less prone to clog pores and not considered comedogenic, not considered to cause skin irritation, and less prone to oxidation in formulations.

Furthermore, the composition restores skin dermal matrix components, increase dermal connective tissue and dermal density by restructuring, re-densifying and revitalizing the dermis for a healthier skin.

The ingredients of the composition may include, but are not limited to, at least one of the following composition formulations discussed herein. The topically applied composition containing Hydrolyzed Plukenetia Volubilis Seed Extract (water based extract of Sacha Inchi) at 2% (w/w) was tested in several in vivo clinical studies on mature female subjects with pronounced saggy skin, deep wrinkles and rougher facial skin texture due to photo-damage and chronological aging. This percentage level was chosen since it provided favorable results further discussed herein, and found to be a commercially acceptable level. The results from the in vivo clinical studies demonstrate that this topical composition containing a water based extract of Sacha Inchi, 2% (w/w) Sacha Inchi is clinically proven to reduce skin sagginess, reshape and sculpt the facial contour, restore skin dermal matrix components, increase dermal connective tissue and dermal density by restructuring, re-densifying and revitalizing the skin.

Measurements were conducted in vivo on human volunteers using the fringe projection system FaceScan® (3D imaging system) and high frequency skin ultrasound Dermascan® C2D image analysis methods. These sophisticated and modern image analysis techniques allow a non-invasive assessment of aged saggy skin and provide quantitative results by scanning facial skin components and morphology before and after the treatment with the composition containing Sacha Inchi. The measurements include facial volume contour and dermis echogenic and non-echogenic surface areas after the treatment containing Sacha Inchi at 2% solution (w/w) in comparison with baseline skin conditions and placebo formulation.

In addition, the composition's further advantages include reduction of facial fine lines and deep wrinkles. The present invention's topically applied composition containing Sacha Inchi at 2% (wt/wt) was tested in a different in vivo clinical study using female subjects with fine lines and deep wrinkles on Crows feet facial area and rougher skin texture. This composition containing Sacha Inchi reduced maximum depth of deepest wrinkle, wrinkle volume and reduced skin roughness parameters such as average roughness, average wrinkle depth and maximum height of the wrinkles for a smoother, softer, younger and healthier looking skin.

Additional potential use and objects of the current invention include sebum regulator and mattifying agent, pore size reducer recommended for oily skin condition. In summary, this cosmetic composition containing Sacha Inchi used in the present invention as described provides anti-sagginess, anti-aging, anti-wrinkle benefits for photo-damaged and chronological aged skin. In addition, the composition containing Sacha Inchi reduces acne scars, blemishes and reduces facial sebum content (area). It also minimizes pore size thereby reducing aspects of an oily skin condition through mattifying and creating an even skin texture.

Furthermore, designed and developed are cosmetic formulations containing a water based extract of Sacha Inchi at 2% solution (w/w) which has been tested during the clinical studies to evaluate skin efficacy of such composition in comparison to a placebo cream. Examples of formulations are included in the description and drawings. Again, these examples are merely given to explain the teachings of the invention, and are not meant to limit the scope of the invention to these particular formulations.

The present invention provides a formulation and method that overcomes past drawbacks tried by others. The present invention provides a water based extract of Sacha Inchi that overcomes several drawbacks of oil based compositions. Oil based compositions have many shortcomings that include, but are not limited to, the oil based compositions may clog pores or be considered comedogenic even in an emulsion form, and oils may be more sensitive to atmospheric oxygen and require to be stabilized with anti-oxidative vitamins or their derivatives that may irritate the skin. The present invention was also analyzed via Gene expression microarray method using full-thickens in vitro skin culture model. This composition containing Sacha Inchi upregulates critical aging genes such as connective tissue growth factor gene (CTGF). CTGF regulates the balance between synthesis and alignment of collagen and elastin fibers, and human longevity master switch gene (FOX03). FOX03 activates upregulation of antioxidant genes. Upregulation includes, for example, in a biological context of gene production a process where a cell increases the quantity of a cellular component, like RNA or protein in response to an external stimulus.

The present invention using Sacha Inchi in a water based extract formulation never used before in the past provides the mechanism to promote these critical aging genes. The mechanism of how this work includes, but is not limited to, for example, the following. During skin aging, the activity of several anti-aging genes is reduced. There are two critical genes involved in the anti-aging process such as, Connective Tissue Growth Factor Gene (CTGF), and Human Longevity Master Switch Gene (FOXO3). CTGF expression is decreasing in aging skin. CTGF gene plays a key role in regulating the production of the extracellular matrix components such as collagen and elastin. CTGF regulates the balance between the synthesis and alignment of collagen and elastin in skin. It is critical for cell adhesion, migration and proliferation of the extracellular matrix organization. Upregulation of the CTGF gene repairs the existing damage in dermis by creating a balance between increasing collagen and elastin production and preventing further disorganization of the protein fibers. FOXO3 gene is a transcription factor, which activates antioxidant genes and protects against extrinsic aging by regulating damaging reactive oxidative species and limiting free radicals accumulation in skin. Using gene expression assays (qPCR method) it was proved, for the first time, that the present invention, a water based extract of Sacha Inchi, in a topical application of Sacha Inchi solution into 3D full-thickness skin tissues increased the expression of the CTGF gene by 133% and the expression of the FOXO3 gene by 66% compared to untreated (control). Sacha Inchi was tested in a solution for the gene expression analysis under typical testing protocols for in vitro/gene expression testing.

The present invention, depending on the particular embodiment, may have applications as a multi-functional composition, and can be use in facial and body anti-aging formulations, facial and body sculpting creams, lotions, serums, gels, masks, facial and body lifting creams, lotions, serums, tonners, gels, masks, anti-sagginess/lifting facial and body daily and night creams, lotions, facial and body reshaping contour formulations, facial lifting color correcting BB and CC creams, lotions, serums, concealers, make up primers, lifting, sunscreens, firming and anti-wrinkles formulations for face and body. The recommended use level is 0.1-10% (w/w) in cosmetic formulations. This recommended use is based on theoretical testing, and actual in vivo testing that has shown these levels are effective. As previously described a level of 2% (w/w) was determined ideal for reasons given herein.

Additional objects of the invention is to use non-animal derived materials, and also contain no genetically modified organisms (GMO) that may contain unknown long term health risks.

It is further an object of the invention to include an effective alternative to surgical techniques such as face lifts, skin grafting, and other invasive facial surgery to reduce severe skin conditions. In addition the composition further includes suitability for all skin types including, but not limited to, users with sensitive skin.

In addition it is an object of the invention to have the composition easy to formulate and manufacture in a simple and convenient method for cost-effective manufacture. It is also an object of the invention to avoid any short shelf life issues.

Another object and advantage of the present invention is to visibly minimize deep wrinkles and fine lines in users. In addition, the composition's advantages further include reduction of facial fine lines, and providing a more even complexion to smoothen skin of the user.

The present invention, depending on the embodiment, may have applications as a multi-active day creams and serums, eye gels, anti-aging night creams and BB/CC creams. Under industry standard typically, one difference between BB and CC creams is CC generally stands for “color correcting” and the products are meant to address issues like redness or swalloness, for example, usually with light-diffusing particles. BB creams are like a lighter foundation with other skin care benefits added. BB creams are short for beauty balms or blemish balms.

The above objects are met by the present invention. In addition the above and yet other objects and advantages of the present invention will become apparent from the hereinafter-set forth Brief Description of the Drawings, Detailed Description of the Invention and claims appended herewith.

These features and other features are described and shown in the following drawings and detailed description.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

So that those having ordinary skill in the art will have a better understanding of how to make and use the disclosed composition and methods, reference is made to the accompanying figures wherein:

FIG. 1 illustrates a prior art rendering of a cross-sectional view of the skin layers in younger skin versus aging skin;

FIG. 2 is a bar graph illustrating the results of Study 1A comparing a placebo with a composition of the present invention for reduction of skin sagginess;

FIG. 3 is a bar graph illustrating the results of Study 1B comparing a placebo with a composition of the present invention for reduction of skin dermis tissue gaps (non-echogenic zones);

FIG. 4 is a bar graph illustrating the results of Study 1C comparing a placebo with a composition of the present invention for increase in skin dermis density (echogenic zones);

FIG. 5 is a 3D FaceScan® illustrating the results comparing a placebo with a composition of the present invention after 28 days of treatment for a subject age 63;

FIG. 6 are ultrasound images of skin dermis illustrating the results comparing a placebo with a composition of the present invention for a subject age 63;

FIG. 7 are ultrasound images of skin dermis illustrating the results comparing a placebo with a composition of the present invention a subject age 60;

FIG. 8 are ultrasound images of skin dermis illustrating the results comparing a placebo with a composition of the present invention a subject age 51;

FIG. 9 is a bar graph illustrating the results of Study 2A comparing a placebo with a composition of the present invention for anti-wrinkle effect showing decrease in maximum depth of deepest wrinkle and wrinkle volume after 21 days of treatment;

FIG. 10 are skin 3D topographic images of the Crow's feet wrinkles of a subject of age 63 illustrating the results comparing the present invention at Day 0 and Day 21 after application of 2% Sacha Inchi;

FIG. 11 is a bar graph illustrating the results of Study 2B comparing a placebo with a composition of the present invention for decrease in skin roughness parameters such as average roughness, maximum wrinkle height and average wrinkle depth;

FIG. 12 are 3D topographic images of the Crow's feet wrinkles for another subject (63 years old) comparing treatment with the present invention at Day 0 and Day 21 after application;

FIG. 13 is a bar graph illustrating the results of Study 3 comparing a placebo with a composition of the present invention for decrease in sebum area and pore size;

FIG. 14 are images of facial sebum spots showing surface area reduction with the present invention at Day 0 and Day 21 after application on a subject (Age 47).

FIG. 15 are images of facial sebum spots showing surface area reduction with the present invention at Day 0 and Day 21 after application on a subject (Age 56).

FIG. 16 are images of facial sebum spots showing surface area reduction with the present invention at Day 0 and Day 21 after application on a subject (Age 45).

### DETAILED DESCRIPTION

In general, the invention overcomes the disadvantages of past attempts to overcome severe skin conditions and works in an entirely different way than prior attempts. Chronological aging, photo-aging, genetics, diet, stress, weight fluctuations or exposure to environmental toxins can cause a gradual loss of connective tissue and dermal matrix components such as collagen, elastin and glycosaminoglycans. Over time, this leads to reduction in skin thickness, loss of dermis density, decrease of orderly deposition and properly alignment of collagen and elastin fibers within the dermal matrix, and ultimately to skin sagginess, laxity, lack of firmness and appearance of fine lines and deep wrinkles and overall aged skin.

Traditionally, such manifestations of aging skin have been addressed with invasive medical and dermatological methods such as cosmetic surgeries, plastic surgeries, facial lifts, chemical acid peels. The invasive methods can lead to skin irritation, inflammation, itching, edema, redness, skin discoloration, discomfort and pain.

The present invention provides for a non-invasive method of treating such skin conditions. Disclosed are hydrolyzed Sacha Inchi proteins and the corresponding peptides tested in topical formulations for skin anti-ageing and beautifying effect.

This topically applied composition containing sustainable non GMO natural peptide Sacha Inchi (Hydrolyzed Plukenetia Volubilis Seed Extract) specifically treats non-invasively these skin conditions that are difficult to treat such as: saggy skin, droopy skin, skin with lack of firmness, elasticity and vigor, skin with laxity, skin with fine lines, deep wrinkles and rougher texture. During in vivo clinical studies conducted on mature female subjects with facial saggy and wrinkled skin, it was surprisingly discovered that the composition containing Sacha Inchi reduced facial volume contour and skin sagginess, reshaped and resculpted the facial contour. Also discovered was that the composition containing Sacha Inchi restored skin dermal matrix components, reduced non-echogenic surfaces in dermis and increased echogenic surfaces, thus increasing dermal connective tissue and dermal density by restructuring, re-densifying and revitalizing the skin.

To our knowledge never before in the cosmetic industry was tested in vivo clinically Sacha Inchi to measure skin anti-sagginess and dermis density benefits. The present invention uses a water based Sacha Inchi extract in a formulation vehicle (cream, and depending on the implementation in other embodiments) so it can be tested directly in vivo on human skin. The focus of the study was primarily the clinical benefit obtained from the non-invasive measurements.

The proposed methods, measurements and bio instruments used to conduct the in vivo clinical studies to determine the efficacy of the Sacha Inchi composition on in vivo human skin was done to assess the cosmetic claims and benefits. Measurements were conducted directly in vivo on human volunteers using the fringe projection system FaceScan® and high frequency skin ultrasound Dermascan® C2D image analysis methods. These sophisticated image analysis techniques allow a non-invasive assessment of aged skin and provide quantitative results by scanning facial skin components and morphology before and after the treatment with the composition containing Sacha Inchi. The measurements include facial volume contour and dermis echogenic and non-echogenic surface areas after the treatment containing Sacha Inchi at 2% solution (w/w) in comparison with baseline skin conditions and placebo formulation. Although 0.1-10% levels of active may be utilized, Sacha Inchi at 2% solution (w/w) was chosen as the test level. This level was chosen because based on our technical experience working with bio-functional actives and considering economic advantages of using such actives at lower levels in topical formulations, a 2% level of Sacha Inchi extract water based for the in vivo clinical studies was considered appropriate.

The present invention may have additional benefits in treating acne and acne scars conditions, skin wounds, burns and scars, razor burns (folliculitis barbae), nicks and cuts caused during and after shaving routines. This invention may have additional benefits in treating dry, xerotic skin and eczema skin conditions, overall improving face and body skin hydration and moisturization.

Also discovered was that this composition containing Sacha Inchi, when tested in a different clinical study, reduced fine lines, deep wrinkles and facial skin roughness parameters such as average roughness (Ra), average wrinkle depth (Rz) and maximum wrinkle height (Rt) for a smoother, softer, younger and healthier looking skin. For this clinical study, PRIMOS Lite® fringe projection image analysis of the skin micro topography was used that allowed quantitative investigation of facial wrinkles and skin roughness texture.

Furthermore, also discovered, while conducting a clinical in vivo study, was that the Sacha Inchi composition has additional benefits on skin as a sebum regulator, mattifying agent, pore size reducing agent, and can be recommended for oily skin condition. This result was very beneficial, and unconventionally not found in other products because of their oil based composition. Since the present invention is a water based extract of Sacha Inchi these additional benefits are possible. In addition, designed and developed were cosmetic formulations containing a water based extract of Sacha Inchi at 2% solution (w/w), which have been tested during the clinical studies to evaluate skin efficacy of such composition in comparison to a placebo cream.

The present invention may have applications as a multi-functional facial and body anti-aging formulations, facial and body sculpting creams, lotions, serums, gels, masks, facial and body lifting creams, lotions, serums, tonners, gels, masks, anti-sagginess/lifting facial and body daily and night creams, lotions, facial and body reshaping contour formulations, facial lifting color correcting BB and CC creams, lotions, serums, concealers, make up primers, lifting, firming and anti-wrinkles formulations for face and body, anti-aging sunscreens formulations, after—sun and self-tanners anti-aging formulations for face and body, skin lighteners, brighteners anti-aging topical formulations for face and body, mists and sprays.

The following definitions are used in this specification and are defined herewith.

Echogenicity—of the tissue refers to the ability to reflect or transmit ultrasounds waves in the context of surrounding tissues. Whenever there is an interface of structures with different echogenicities, a visible difference in contrast will be apparent on the screen. Based on echogenicity, a structure can be characterized as hyperechoic (white on the screen), hypoechoic (gray on the screen) and anechoic (black on the screen).

Echogenic=Substances that are capable of producing echos and reflecting sound waves. In the dermis the echogenicity is given by the density of the connective tissue components. An increase in the echogenic zones means an increase in dermis density.

Non-echogenic=Substances that cannot produce echos and cannot reflect sound waves. In the dermis the non-echogenic zones lack of connective tissue and show numerous tissue gaps. A decrease in the non-echogenic zones means a decrease in the tissue gaps in the dermis.

Maximum Depth of the Deepest Wrinkle=measures the deepest depth of the deepest wrinkle from the Crow's feet area. A decrease in this parameter characterizes less deep wrinkles, thus an anti-wrinkle effect.

Wrinkle Volume represents the average wrinkle volume. A decrease in this parameter characterizes an anti-wrinkle effect.

Average roughness (Ra) represents the average roughness of all heights from the peaks and valleys of the wrinkle profile. A decrease indicates a smoothing effect.

Average wrinkle depth (Rz) represents the average depth of 5 consecutive sections of the wrinkle profile (the difference between the highest peak to lowest valley). A decrease indicates reduction of average wrinkle depth, thus an anti-wrinkle effect.

Maximum wrinkle height (Rt) represents the greatest height of the wrinkle profile or highest peak of the wrinkle profile. A decrease indicates reduction of maximum wrinkle height, thus an anti-wrinkle effect.

Connective Tissue Growth Factor Gene-CTGF this gene is critical for cell adhesion, migration & proliferation for ECM organization. Regulates balance between synthesis & alignment of collagen & elastin. Orchestrates repair & prevention of further dermal elastosis.

Human Longevity Master Switch Gene—FOXO3—is a transcription factor protects against extrinsic aging. It regulates protein homeostasis network. It also activates upregulation of antioxidant genes.

Clinical studies were performed testing, among other things, facial volume contour, restructuring and re-densifying effect, anti-wrinkle effect and skin roughness. The features and benefits of the Sacha Inchi composition include at least the following. The extracted peptides from its seed contain predominately small peptides as compared to other currently available products. The sustainable non GMO water based peptides of Sacha Inchi or the hydrolyzed Plukenetia Volubilis seed extract are accomplished via a biotechnological manufacturing process that uses an exotic botanical source of a Peruvian superfood seed. The benefits of the present invention further includes reduction of facial sagginess for more contoured skin, restructuring of the skin by increasing dermal density, decreasing roughness for smoother skin and helping reduce wrinkles. These benefits are accomplished due to, among other things, the surprising effect of the Sacha Inchi on various aging gene factors further described herein. These overall anti-aging benefits are accomplished by the water based extract of Sacha Inchi's unique chemical composition (amino acids and small peptides) and low average molecular weight, which ensure optimal delivery of Sacha Inchi into skin epidermis and dermis. This delivery of Sacha Inchi's specific amino acids and natural peptides into skin epidermis and dermis activates the expression of critical anti-aging genes such as CTGF and FOXO3. Specifically, the activation of the CTGF gene stimulates the proper organization and synthesis of the extracellular matrix components (ECM) to enhance its anti-aging performance, by restructuring dermal density, reducing skin sagginess and laxity, reducing wrinkles and smoothing skin texture. The activation of the FOXO3 gene protects against extrinsic aging by regulating damaging reactive oxidative species and limiting free radicals accumulation in skin.

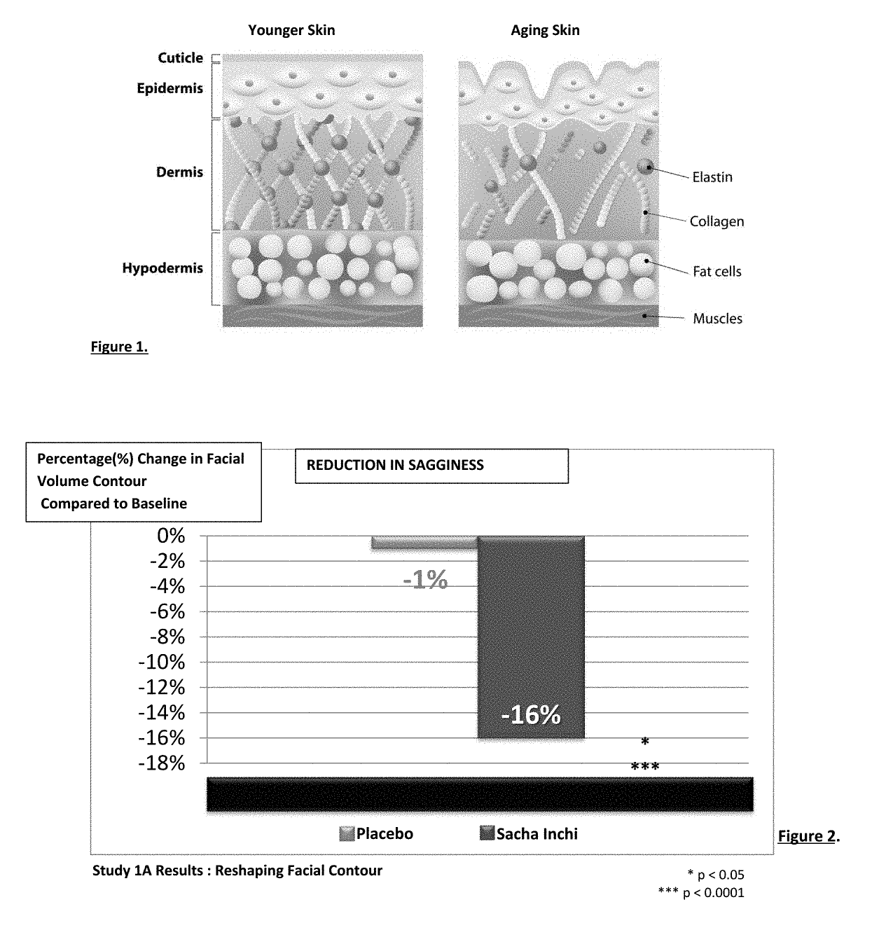

Adverting to FIG. 1, shown is a cross sectional illustration of skin. The skin's dermal layer derives its strength and resiliency from the unique properties of two key structural proteins-collagen and elastin.

The skin aging is affected by collagen and elastin. Namely it decreases in synthesis and the decrease of the proteins accelerates degradation.

Also there is disorderly deposition and misalignment within the dermal matrix (dermal elastosis). These changes contribute to the formation of wrinkles, sagginess and fragility within the skin.

Gene expression protocol below was used during the studies shown in the following examples. Gene expression assay was performed on full thickness in vitro skin culture model (EFT-400, MatTek) following 24 hour exposure to the test materials. Culture media with Sacha Inchi was tested against culture media with control (aqueous vehicle). Sacha Inchi was tested at 4% concentration. Gene expression analysis was performed with a Life Technologies QuantStudio 12K Flex instrument and qPCR reactions were run using validated TaqMan® Gene Expression Assays for CTGF and FOXO3 genes in an OpenArray® or 96-well format. The testing showed, among other things, that the water based extract of Sacha Inchi's specific amino acids and natural peptides activates the expression of critical anti-aging genes such as CTGF and FOXO3.

Overview of the Clinical Studies done to test the composition of the present invention include the following:

Studies 1 A & B:
Anti-Sagginess/Reshaping and restructuring effect
Reduction in sagginess
Reduction in dermis tissue gaps
Increase in dermis density

Studies 2 A & B:
Anti-wrinkle Effect
Decrease in maximum depth of deepest wrinkle and wrinkle volume
Decrease in skin roughness

The following examples are given to merely to illustrate the features of the present invention and in no means are intended to limit the scope of the invention to these specific examples.

Study 3: Sebum Surface Area and Pore Size Measurement
reduction in sebum surface area
reduction in pore size

The study protocol for Study 3 included the following.

The following methods and instruments were used in the testing. Tested were 9 females and males volunteers (26-65 years old) with oily skin face and large pore size. Water based extract of Sacha Inchi at 2% (MG1-31A) formula and a placebo cream (MG1-31B) were used on half-face on randomized manner for a duration of 21 days and at a dosage of 5 mg/cm². The instruments used were Visioscan® VC98, Sebufix® F16 and Visiopor®PP39N (Courage & Khazaka). Measurements of sebum surface area and pore size were taken at baseline (D0) and after 21 days of treatment with water based extract of Sacha Inchi and placebo creams. Facial sebum surface area images were taken with Visioscan®VC 98 and Sebufix® F16. Visioscan®, VC 98 is a UVA-light video camera with high resolution to study the skin surface directly. The images show the structure of the skin. Sebufix® F16 is a special foil absorbing the sebum of the skin surface due to its micro pores and showing them as spots in different sizes. When mounted on the Visioscan® camera, the qualitative sebum production can be monitored in real time. Furthermore, Visioscan® VC 98 is special UVA-light video camera with high resolution that provides image analysis of skin surface topography. The Visioscan VC98 is suitable to evaluate Sebufix®F16 foils for the qualitative and quantitative sebum production. With Sebufix®F16, measurement of the activity of each sebum gland in the area of interest is visible in real time. In the images sebum is visible as black spots. Visiopor PP39N uses a specific UVA light camera to analyze the pore size. Pore sizes were measured with Visiopor® PP39N. Subjects discontinued using any other cosmetic products on the face with 3 days before the study and for the duration of the study.

### Example 1

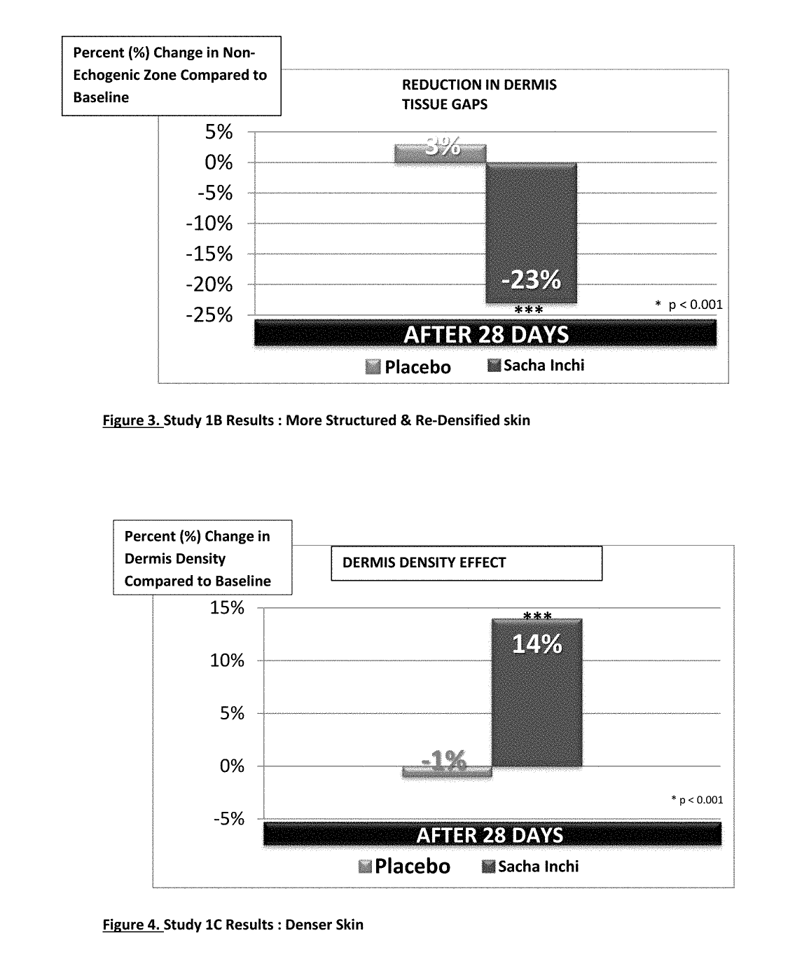

Study 1A and 1B involved testing the present invention for reshaping and restructuring effect. Results of this testing are shown in FIG. 2 and FIG. 3 respectively.

The following material and dosages were used in the testing. Tested were half-face (15) female volunteers (50-65 years old) with facial skin sagginess on jawlines. A Placebo cream was used and the placebo cream+2% water based extract of Sacha Inchi. The dosage and duration was 5 mg/cm² two applications per day for 28 days. The instruments used were FaceScan® (3D Face Scanning Imaging) and Dermascan C®2D (Ultrasound Imaging). The testing methods included a single blinded half-face randomized intra-individual study. A time limit of 72 hours before and throughout the duration of the study, volunteers stopped using any cosmetic products on their face. Measurements were taken at baseline (day 0) and after 28 days (day 28). The test conditions were Room Temperature: 21° C.±2° C.; Relative Humidity: 30±10%.

The formulation for clinical Study 1 included the following:

| | | Sacha Inchi | Placebo |
|---|---|---|---|
| | | MG1-58A | MG1-58B |
| INCI Name/Chemical | | Percentage | Percentage |
| Name | Trade Name | (%) | (%) |
| Phase A | | | |
| Water | | 86.70 | 88.70 |
| Carbomer | | 0.30 | 0.30 |
| Glycerin | | 2.00 | 2.00 |
| Disodium EDTA | | 0.05 | 0.05 |
| Phase B | | | |
| Cetearyl Alcohol and | | 2.75 | 2.75 |
| Ceteareth 20 | | | |
| Caprylic Capric | GalMol CCT | 1.50 | 1.50 |
| Triglycerides | | | |
| Isopropyl Palmitate | | 3.00 | 3.00 |
| Isodecyl Neopentanoate | | 0.50 | 0.50 |
| Phase C | | | |
| Triethanolamine | | 0.20 | 0.20 |
| Phase D | | | |
| Hydrolyzed Plukenetia | Sacha Inchi | 2.00 | |
| Volubilis Seed Extract | | | |
| Phenoxyethanol, | | 1.00 | 1.00 |
| Ethylhexylglycerin | | | |

Shown in FIG. 2 is the reshaping facial contour result. A decrease of 16% with the present invention versus 1 percent using the placebo is shown. By reducing skin sagginess, the water based extract of Sacha Inchi helps reshape the jawline and facial contour for a more sculpted younger and healthier looking appearance.

Shown in FIG. 3 are the results for the structured and redensified skin test. A reduction of 23% using the present invention versus an increase of 3% with the placebo is shown. The present invention restructures skin by decreasing tissue gaps in the dermis for healthier rejuvenated skin.

FIG. 4 illustrates the results for denser skin. The results show a 14% increase in dermis density using the present invention. This result is versus a 1% decrease in dermis density with the placebo facial cream. The present invention redefines skin contour by significantly increasing the density of the dermis for firmer cheeks and jawlines.

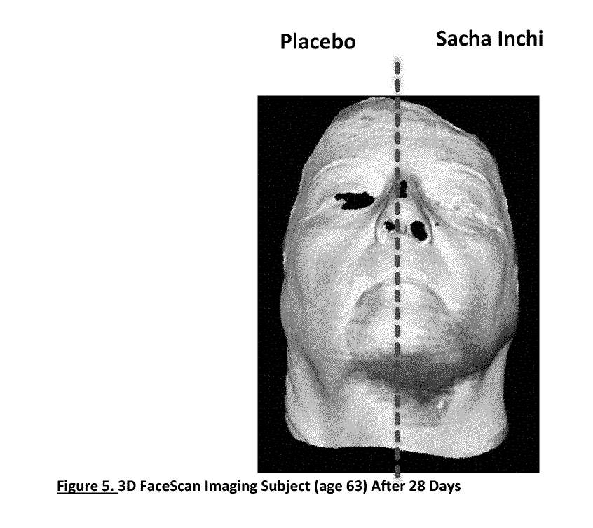

FIG. 5 illustrates a 3D scan for a subject aged 63. Half of the face was treated with a placebo cream and the other half of the subject's face was treated with a facial cream of the present invention. After 28 days this subject had a 45% decrease in skin sagginess after the water based extract of Sacha Inchi treatment. In the 3D FaceScan® image a decrease in the facial sagginess volume represents improvement in the facial contour. The decrease in facial sagginess volume is seen in 3D images in blue color.

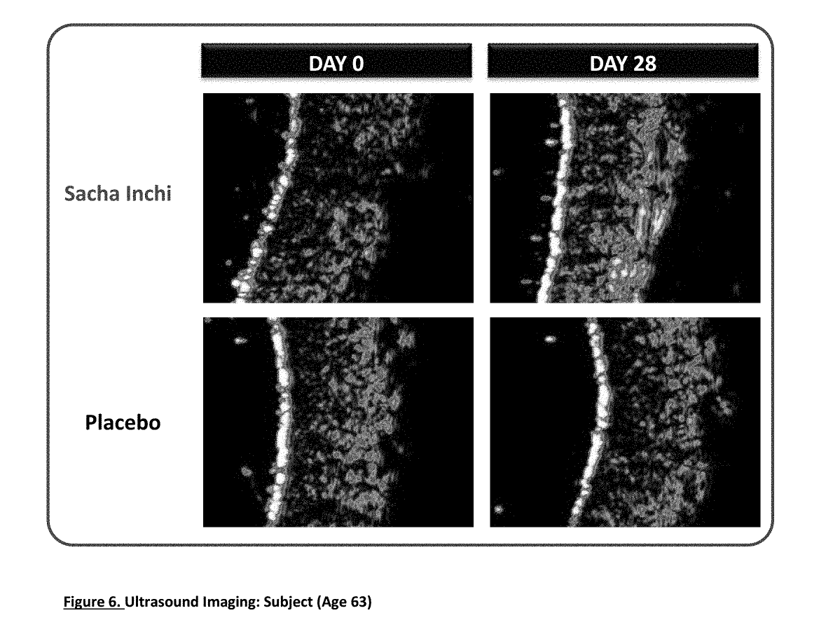

The same test subject is also shown in FIG. 6, subject (age 63). This figure illustrates skin ultrasound imaging. After 28 days, this subject had a 47% decrease in tissue gaps and a 49% increase in dermis density with the present invention for healthier skin. In the ultrasound images the echogenic zones (such as connective tissue) appear colored (from green to yellow) while the non-echogenic tissues (such as tissue gaps) appear in black.

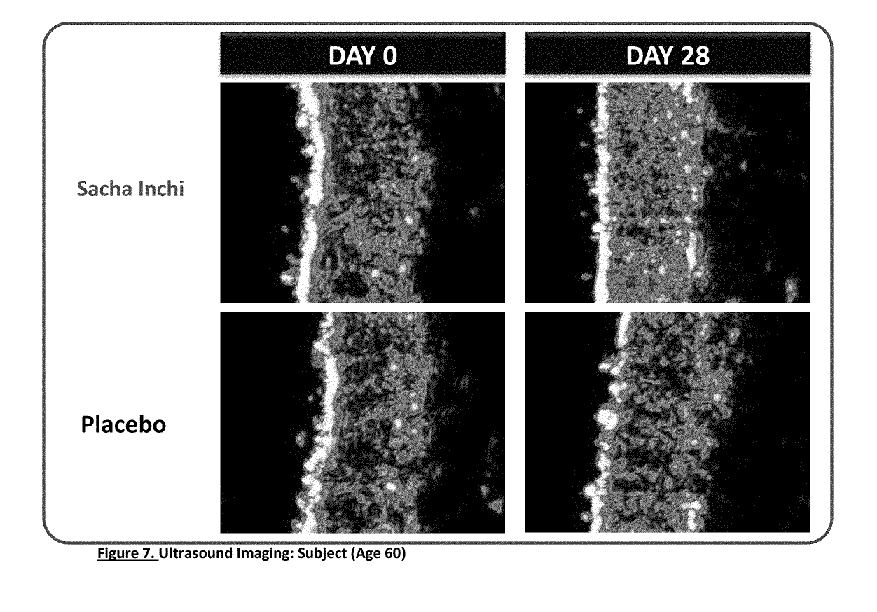

In FIG. 7, ultrasound imaging is shown for another Subject (age 60). After 28 days, this subject had a 50% reduction in tissue gaps with the present invention, the water based extract of Sacha Inchi, for denser dermis and rejuvenated skin. In the ultrasound images the echogenic zones (such as connective tissue) appear colored (from green to yellow) while the non-echogenic tissues (such as tissue gaps) appear in black.

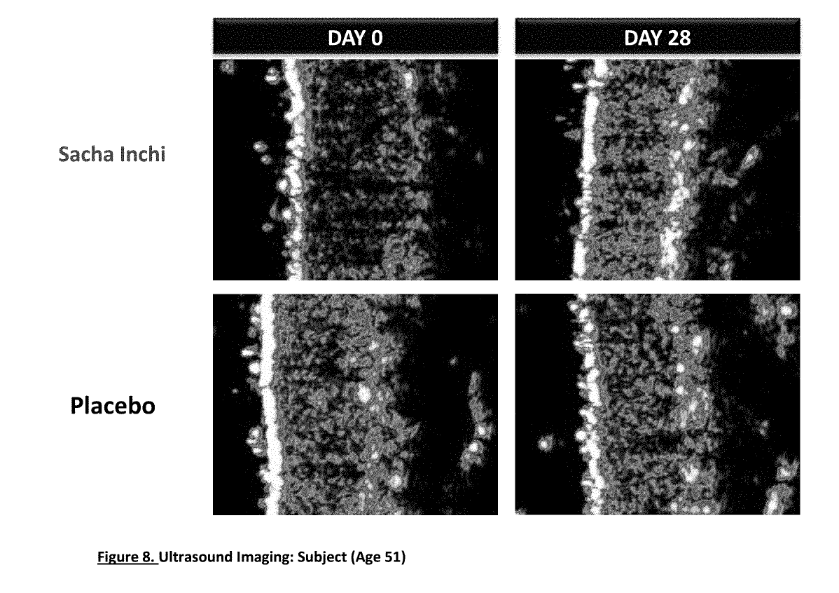

In FIG. 8, ultrasound imaging is shown for a different Subject (age 51). The water based extract of Sacha Inchi increased dermal density by 41% in this subject for more restored, revitalized and healthier looking skin.

In the ultrasound images the echogenic zones (such as connective tissue) appear colored (from green to yellow) while the non-echogenic tissues (such as tissue gaps) appear in black. In the 3D FaceScan® image a decrease in the facial sagginess volume represents improvement in the facial contour. The decrease in facial sagginess volume is seen in 3D images in blue color.

### Example 2

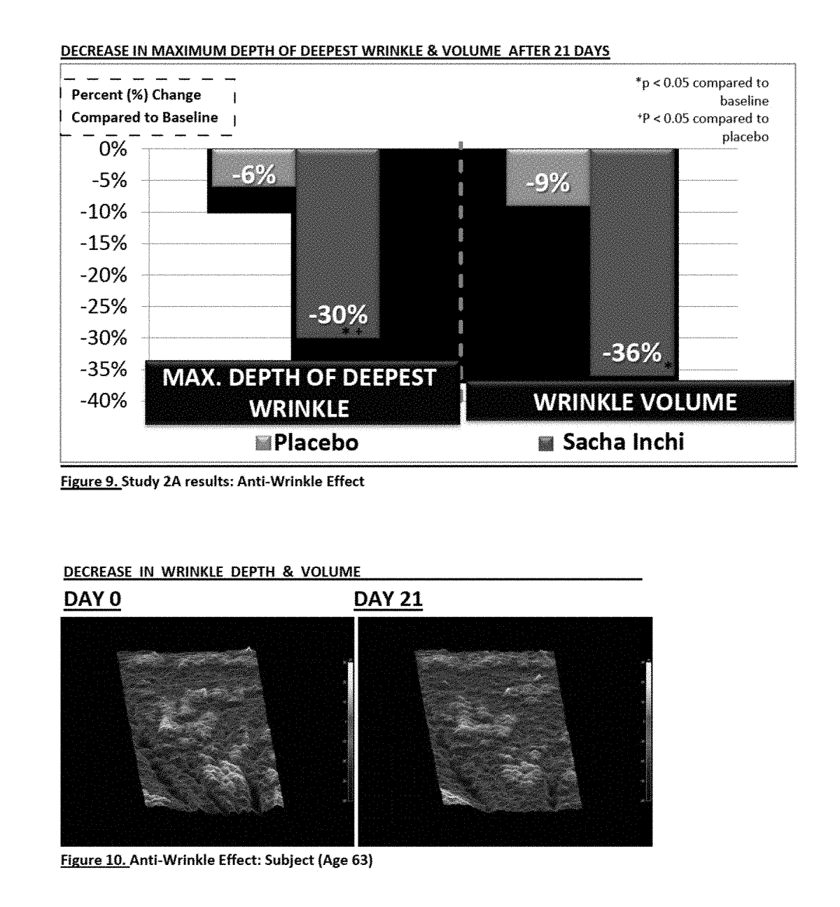

Study 2 as shown in FIGS. 9-12 dealt with the anti-wrinkling effect. For Study 2, the procedure included the following steps. Skin site volunteers had Crow's fee area tested. Seven (7) females (28-65 years old) were tested. The test subject had fine lines and pronounced wrinkles. A placebo cream was applied and Placebo Cream+2% water based extract of Sacha Inchi. The dosage and duration of the test was 5 mg/cm² twice daily for 21 days. Instruments used to test the results included PRIMOS® Lite. The parameters tested were skin roughness, maximum depth of the deepest wrinkles and volume. The treatment method included a single blinded half-face randomized intra-individual study. 72 hours before and throughout the duration of the study, volunteers stopped using any cosmetic products on their face. The test conditions were Room Temperature: 21° C.±2° C.; Relative Humidity: 30±10%.

The formulation for clinical Study 2 included the following:

| | | Sacha Inchi | Placebo |
|---|---|---|---|
| | | Mg1-31A | MG1-31B |
| INCI Name/Chemical | | Percentage | Percentage |
| Name | Trade Name | (%) | (%) |
| Phase A | | | |
| Water | | 85.90 | 87.90 |
| Carbomer | | 0.30 | 0.30 |
| Glycerin | | 2.00 | 2.00 |
| Disodium EDTA | | 0.05 | 0.05 |
| Phase B | | | |
| Cetearyl Alcohol and | | 2.50 | 2.50 |
| Ceteareth 20 | | | |
| Squalane | OleaClear Olive | 4.00 | 4.00 |
| | Squalane | | |
| Isopropyl Palmitate | | 2.00 | 2.00 |
| Phase C | | | |
| Triethanolamine | | 0.25 | 0.25 |
| Phase D | | | |
| Hydrolyzed Plukenetia | Sacha Inchi | 2.00 | |
| Volubilis Seed Extract | | | |
| Phenoxyethanol, | | 1.00 | 1.00 |
| Ethylhexylglycerin | | | |

Shown in FIG. 9 is the Anti-Wrinkle effect of the present invention. After 21 days the maximum depth of the deepest wrinkle decrease 30% and wrinkle volume decrease 36% using the facial cream of the present invention as compared to 6% and 9% with a placebo cream, respectively. Water based extract of Sacha Inchi reduces wrinkles after 21 days to reveal more youthful skin for ageless beauty.

Shown in FIG. 10 is the anti-wrinkle effect for a subject (age 63). Compared is the present invention at day 0 and at Day 21 after application. Again a 2% water based extract of Sacha Inchi was applied. After 21 days, the water based extract of Sacha Inchi decreased the maximum depth of the deepest wrinkle by 32% and wrinkle volume by 18% revealing smoother skin.

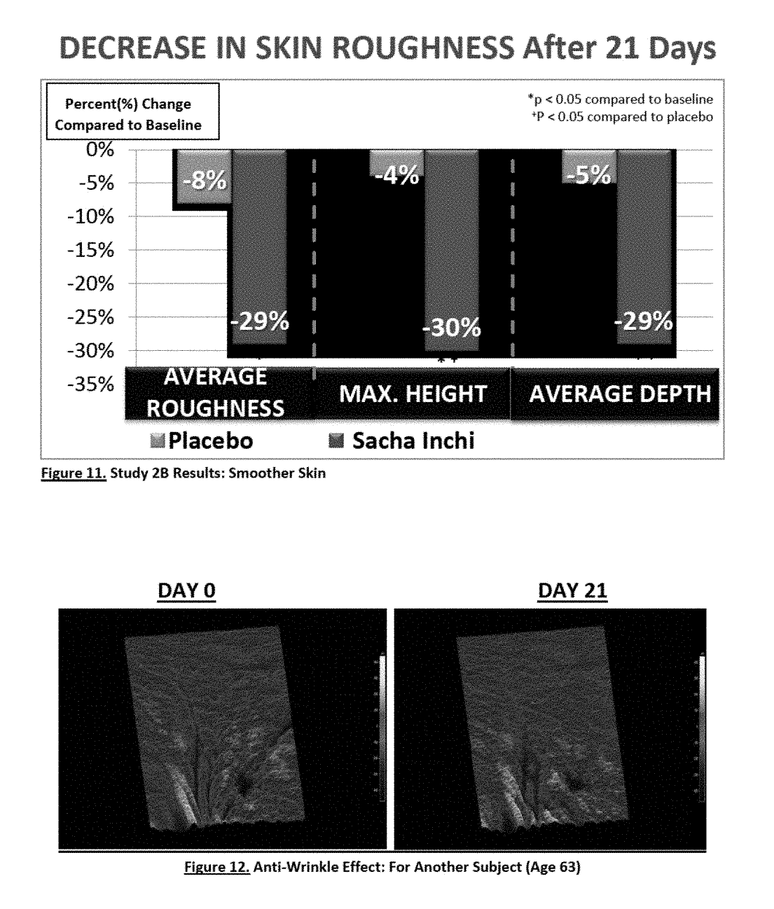

Shown in FIG. 11, is the decrease in skin roughness after 21 days of application with the present invention. Smoother skin resulted with use of the present invention on the test subject. After only 21 days, the water based extract of Sacha Inchi decreased skin roughness more than 20% in 3 parameters compared to the placebo for softer, smoother skin.

Both Clinical Studies Showed that the Present Invention:
Reduces facial sagginess for more contoured skin
Restructures skin by increasing dermal density
Decreases roughness for smoother skin
Helps reduce wrinkles

The ingredients included Hydrolyzed Plukenetia Volubilis Seed Extract. The suggested application are:
Anti-aging formulations
Sculpting creams & serums
Make-up primers
BB/CC creams
Lifting, firming & anti-wrinkle formulations

And suggested use level: 0.1-10%

### Example 3

For Clinical Study 1 a further composition and procedure of making is given as shown below for exemplary reasons.

| INCI Name/Chemical | | | Percentage | Percentage |
|---|---|---|---|---|
| Name | Trade Name | Supplier | (%) | (%) |
| Phase A | | | MG1-58A | MG1-58B |
| | | | | (Placebo) |
| Water | Water | | 72.00 | 74.00 |
| Carbomer | Carbopol 980 (2% | Lubrizol | 15.00 | 15.00 |
| | Solution) | | | |
| Glycerin | Glycerin | | 2.00 | 2.00 |
| Disodium EDTA | Versene ™ Na2 | The Dow | 0.05 | 0.05 |
| | Crystals | Chemical | | |
| | | Company | | |
| Phase B | | | | |
| Cetearyl Alcohol and | Jeecol CS | | 2.75 | 2.75 |
| Ceteareth 20 | | | | |
| Caprylic Capric | GalMol CCT | TRI-K | 1.50 | 1.50 |
| Triglycerides | | Industries, Inc. | | |
| Isopropyl Palmitate | Isopropyl Palmitate | | 3.00 | 3.00 |
| Isodecyl Neopentanoate | Isodecyl | | 0.50 | 0.50 |
| | Neopentanoate | | | |
| Phase C | | | | |
| Triethanolamine | Triethanolamine 99% | Jeen | 0.20 | 0.20 |
| | | International | | |
| | | Corporation | | |
| Phase D | | | | |
| Hydrolyzed Plukenetia | Sacha Inchi | TRI-K | 2.00 | 0.00 |
| Volubilis Seed Extract | | Industries | | |
| Phenoxyethanol, | Euxyl ® PE 9010 | Schulke | 1.00 | 1.00 |
| Ethylhexylglycerin | | | | |

### Procedure:

1) Add Carbopol to the water, mix well until completely hydrated. Heat to 70° C. and add remaining ingredients of Phase A.
2) Combine Phase B ingredients in a separate beaker and heat to 70-75° C. Add Phase B to Phase A and mix well until uniform.
3) At 70° C. add Phase C and homogenize for 3 minutes. Switch to propeller mixer and continue mixing.
4) Cool batch to 40° C. Add Phase D ingredients one at the time and mix until uniform.
5) Cool batch to 25° C. and adjust for water loss.

### Example 4

In Example 4 for Clinical Study 2 a further composition and procedure of making is given as shown below for exemplary reasons. This formulation was also used in Clinical Study 3 for a facial cream as a Sebum regulator and pore size minimizer formula.

| INCI Name/Chemical | | | Percentage | Percentage |
|---|---|---|---|---|
| Name | Trade Name | Supplier | (%) | (%) |
| Phase A | | | MG1-31A | MG1-31B |
| | | | | (Placebo) |
| Water | Water | | 71.20 | 73.20 |
| Carbomer | Carbopol 980 (2% | Lubrizol | 15.00 | 15.00 |
| | Solution) | | | |
| Glycerin | Glycerin | | 2.00 | 2.00 |
| Disodium EDTA | Versene ™ Na2 | The Dow | 0.05 | 0.05 |
| | Crystals | Chemical | | |
| | | Company | | |
| Phase B | | | | |
| Cetearyl Alcohol and | Jeecol CS | | 2.50 | 2.50 |
| Ceteareth 20 | | | | |
| Squalane | OleaClear Olive | TRI-K | 4.00 | 4.00 |
| | Squalane | Industries, Inc. | | |
| Isopropyl Palmitate | Isopropyl Palmitate | | 2.00 | 2.00 |
| Phase C | | | | |
| Triethanolamine | Triethanolamine 99% | Jeen | 0.25 | 0.25 |
| | | International | | |
| | | Corporation | | |
| Phase D | | | | |
| Hydrolyzed Plukenetia | Sacha Inchi | TRI-K | 2.00 | 2.00 |
| Volubilis Seed Extract | | Industries | | |
| Phenoxyethanol, | Euxyl ® PE 9010 | Schulke | 1.00 | 1.00 |
| Ethylhexylglycerin | | | | |

### Procedure:

1) Add Carbopol to the water, mix well until completely hydrated. Heat to 70° C., then add remaining ingredients of Phase A.
2) Combine Phase B ingredients and heat to 70-75° C. Add Phase B to Phase A, mix well.
3) Add Phase C, homogenize for 3 minutes. Switch to propeller mixer and continue mixing
4) Cool batch to 40° C. Add Phase D ingredients one at the time and mix until uniform.
5) Cool batch to 25 C and adjust for water loss.

Example 4 also demonstrates anti wrinkles facial cream used in Clinical Study 2 for anti-wrinkle claims and reducing skin roughness. In addition it may be a facial cream for a sebum regulator and pore size minimizer Shown as an example is FIG. 12. FIG. 12 illustrates a decrease in wrinkle depth and volume after treatment with 2% water based extract of Sacha Inchi. Images are shown for another representative Subject (63 years old). Image Analysis 3D pictures of Crow's feet wrinkles were taken before (Day 0) and after 21 days of treatment (D21) as shown in FIG. 12.

For this subject, it was reported a 43% decrease in wrinkle volume and 24% decrease in wrinkle depth compared to baseline after 21 days of treatment measured with PRIMOS^{Lite®} 3D fringe projection image analysis technique.

### Example 5

Shown for this Example 5 is an exemplary description of a Zero Gravity Face and Neck Cream.

| INCI Name/Chemical | | | Percentage |
|---|---|---|---|
| Name | Trade Name | Supplier | (%) |
| Phase A | | | |
| Water | Water | | 70.20 |
| Disodium EDTA | Versene NA2 | The Dow | 0.05 |
| | Crystals | Chemical | |
| Xanthan Gum | Vanzan ® NF-C | R. T. Vanderbilt | 0.25 |
| | | Company | |
| Magnesium Aluminum Silicate | Veegum ® ULTRA | Vanderbilt | 0.50 |
| | Granules | Minerals, LLC | |
| Glycerin | Glycerin 99.7% | Bruchem, Inc. | 3.00 |
| | Natural USP | | |
| Phase B | | | |
| Glyceryl Stearate, Cetearyl Alcohol, | TRIastin | TRI-K | 3.50 |
| Sodium Stearoyl Lactylate | | Industries, Inc. | |
| Glyceryl Stearate, PEG-100 Stearate | Jeechem GMS-165 | Jeen ® | 2.00 |
| | | International | |
| Caprylic/Capric Triglyceride | Jeechem CTG | Jeen ® | 4.00 |
| | | International | |
| Squalane | OleaClear Olive | TRI-K | 3.00 |
| | Squalane | Industries, Inc. | |
| C12-15 Alkyl Benzoate | Finsolv ® TN | Innospec | 3.00 |
| | | Performance | |
| | | Chemicals | |
| Theobrama Cacao (Cocoa) | Tomasa Cocoa B | TRI-K | 2.00 |
| Seed Butter | | Industries, Inc. | |
| Dimethicone | XIAMETER ® | Xiameter/nexeo ™ | 2.00 |
| | DPMX-200 Silicone | solutions | |
| | Fluid 350 | | |
| Caprylyl Methicone | Dow Corning ® | Xiameter/nexeo ™ | 2.00 |
| | FZ-3196 | solutions | |
| Behenyl Alchol | Behenyl Alcohol | The Herbarie | 1.00 |
| Tocopheryl Acetate | Vitamin E Acetate | TRI-K | 0.50 |
| | | Industries, Inc. | |
| Phase D | | | |
| Benzyl Alcohol, Water, Potassium | Tristate E | TRI-K | 1.00 |
| Sorbate, Sodium Benzoate | | Industries, Inc. | |
| Hydrolyzed Plukenetia | Sacha Inchi | TRI-K | 2.00 |
| Volubilis Seed Extract | | Industries, Inc. | |

Procedure:

1. Heat water to 60° C., dissolve Disodium EDTA.

2. Disperse remaining ingredients of Phase A one at a time until fully hydrated and uniform.

3. Heat to 75° C. Combine and heat Phase B to 75° C.

4. Add Phase B to Phase A, homogenize for 3 minutes. Start cooling the batch with side sweep mixing.

5. Add Phase D ingredients one at a time to batch at or below 40° C.

6. Cool batch to 30° C.

Hydrolyzed Plukenetia Volubilis Seed Extract for forming a zero gravity face and neck cream may or may not be added, depending on the embodiment as an ingredient of Phase D.

### Example 6

Shown for this Example 6 is an exemplary description of a Scuplting Face Neck Serum.

| INCI Name/Chemical | | | Percentage |
|---|---|---|---|
| Name | Trade Name | Supplier | (%) |
| Phase A | | | |
| Water | Water | | 90.10 |
| Glycerin | Glycerin | | 2.00 |
| Disodium EDTA | Versene ™ Na2 | The Dow | 0.05 |
| | Crystals | Chemical | |
| | | Company | |
| Panthenol | DL Panthenol | TRI-K | 0.50 |
| | FCC/USP | Industries, Inc. | |
| Sodium Polyacrylate, Dimethicone, | Dow Corning ® RM | Dow Corning | 2.25 |
| Cyclopentasiloxane, Trideceth-6, | 2051 Thickening | Corporation | |
| PEG/PPG 18/18 Dimethicone | Agent | | |
| Phase B | | | |
| Squalane | OleaClear Olive | TRI-K | 2.00 |
| | Squalane | Industries, Inc. | |
| Tocopheryl Acetate | Vitamin E | TRI-K | 0.10 |
| | Acetate | Industries, Inc. | |
| Hydrolyzed Plukenetia | Sacha Inchi | TRI-K | 2.00 |
| Volubilis Seed Extract | | Industries, Inc. | |
| Phenoxyethanol, | euxyl ® PE 9010 | Schülke & Mayr | 1.00 |
| Ethylhexylglycerin | | GmbH | |

Procedure:
1. Add Phase A ingredients individually in the given order. Ensure uniformity before each addition.
2. Add Phase B ingredients individually. Mix until uniform.

### Formula Properties: pH: 5.65

### Example 7

Shown for this Example 7 is an exemplary description of a Revitalizing Clay Face Firming Mask.

| INCI Name/Chemical | | | Percentage |
|---|---|---|---|
| Name | Trade Name | Supplier | (%) |
| Phase A | | | |
| Water | Water | | 58.52 |
| Magnesium Aluminum Silicate | Veegum Ultra | Vanderbilt Minerals | 4.50 |
| Phase B | | | |
| Propanediol | Zemea | Dupont Tate & Lyle | 2.50 |
| | | Bioproducts | |
| Glycerin | Glycerin | | 2.50 |
| Xanthan Gum | Vanzan NF-C | Vanderbilt Minerals | 0.24 |
| Phase C | | | |
| Illite | Jarxiotic ™ RC-NS | Jarchem | 2.00 |
| | | Industries, Inc. | |
| Kaolin | ImerCare ™ 04K | IMERYS North | 24.00 |
| | | America | |
| Silica | SYLOID ® 244FP | W. R. Grace & | 1.40 |
| | | Co.-Conn | |
| Phase D | | | |
| Phenoxyethanol | Tristat P5 | TRI-K | 0.90 |
| | | Industries, Inc. | |
| Phase E | | | |
| Water, Rosa Damascena | Active Water | TRI-K | 0.50 |
| extract, citric acid, sodium benzoate, | Damask Rose | Industries, Inc./ | |
| potassium sorbate | | Phenbiox | |
| Water, Basilico extract, citric acid, | Active Water Basil | TRI-K | 0.50 |
| sodium benzoate, potassium sorbate | | Industries, Inc./ | |
| | | Phenbiox | |
| Hydrolyzed Plukenetia | Sacha Inchi | TRI-K | 2.00 |
| Volubilis Seed Extract | | Industries, Inc. | |
| Phase F | | | |
| Polysorbate 20 | Polysorbate 20 | Spectrum Chemical | 0.30 |
| | | Manufacturing Corp. | |
| Fragrance | Aromatic Rose | | 0.14 |
| | Extract | | |

Procedure:
1. Charge water to main. Begin mixing on propeller.
2. Sprinkle in Veegum. Begin heating to 65-70° C. In order to fully hydrate Veegum.
3. Mix 10-15 minutes. Turn off heat.
4. Premix phase B. Add to main.
5. Add phase C one at a time. Mix until uniform.
6. Add phase D. Mix until uniform.
7. Add phase E one at a time. Mix until uniform.
8. Premix phase F. Add to main. Mix until uniform.
   Formula Properties: pH 4.98 Viscosity (T-D@5 rpm) 124,000 cps

### Example 8

Shown for this Example 8 is an exemplary description of a composition for reducing sebum surface area and pore size.

In one embodiment, below is an exemplary description of a Revitalizing Clay Face Firming Mask.

| INCI Name/Chemical | | | Percentage |
|---|---|---|---|
| Name | Trade Name | Supplier | (%) |
| Water | Water | | 70.20 |
| Disodium EDTA | Versene NA2 | The Dow Company | 0.05 |
| | Crystals | | |
| Xantham Gum | Vanzan ® NF-C | R. T. Vanderbilt | 0.25 |
| | | Company | |
| Magnesium Aluminum Silicate | Veegum Ultra | Vanderbilt Minerals, | 0.50 |
| | Granules | LLC | |
| Glycerin | Gylcerin 99.7% | Bruchem, Inc. | 3.00 |
| | Natural USP | | |
| Phase B | | | |
| Glyceryl Stearate, Ceteary Alchol, | TRIsatin | TRI-K | 3.50 |
| Sodium Stearoyl Lactylate | | Industries, Inc. | |
| Glyceryl Stearate, PEG-100 Stearate | Jeechem GMS-165 | Jeen ® | 2.00 |
| | | International | |
| Caprylic/Capric Triglyceride | Jeechem CTG | Jeen ® | 4.00 |
| | | International | |
| Squalane | OleaClear Squalane | TRI-K | 3.00 |
| | | Industries, Inc. | |
| C12-C15 Alkyl Benzoate | Finsolv ® TN | Innospec Performance | 3.00 |
| | | Chemicals | |
| Theobroma Cacao (Cocoa) | Tomasa Cocoa B | TRI-K | 2.00 |
| Seed Butter | | Industries, Inc. | |
| Dimethicone | XIAMTER ® | Xiameter/nexeo ™ | 2.00 |
| | DPMX-200 | Solutions | |
| | Silicone Fluid 350 | | |
| Caprylyl Alcohol | Dow Corning ® | Xiameter/nexeo ™ | 2.00 |
| | FZ-3196 | Solutions | |
| Behenyl Alcohol | Behenyl Alcohol | The Herbarie | 1.00 |
| Tocopheryl Acetate | Vitamin E Acetate | TRI-K | 0.50 |
| | | Industries, Inc. | |
| Phase D | | | |
| Benzyl Alcohol, Water, | Trisat E | TRI-K | 1.00 |
| Potassium Sorbate, | | Industries, Inc. | |
| Sodium Benzoate | | | |
| Hydrolyzed Plukenetia | Sacha Inchi | TRI-K | 2.00 |
| Volubilis Seed Extract | | Industries, Inc. | |

### Procedure:

1. Heat water to 60° C., dissolve Disodium EDTA.
2. Disperse remaining ingredients of Phase A one at a time until fully hydrated and uniform. Heat to 75° C.
3. Combine and heat Phase B to 75° C.
4. Add Phase B to Phase A, homogenize for 3 minutes. Start cooling the batch with side sweep mixing.
5. Add Phase D ingredients one at a time to batch at or below 40° C.
6. Cool batch to 30° C.

Clinical Study 3: Sebum Surface Area and Pore Size Measurement

Study 3 involved testing the present invention in vivo for sebum area and pore size reduction. Tested were half-face (9) nine females and males volunteers (26-65 years old) with oily facial skin condition. A placebo cream (MG1-31B) was used and 2% water based extract of Sacha Inchi (MG31A formula). The dosage was 5 mg/cm2 and for a duration of 21 days with two applications per day.

The instruments used were: Visioscan®VC 98, Sebufix®F16 and Visiopor®PP39N. This was a single blinded half-face randomized intra-individual study. 72 hours before the study and during the study panelists stopped using any cosmetic products on their face. Measurements of sebum surface area and pore size were performed at baseline at day zero (D0) and after 21 days of treatment (D21) on facial skin sites treated with water based extract of Sacha Inchi Cream and Placebo Cream. Facial sebum spots surface area images were taken with the Visioscan VC 98 and Sebufix F16 instruments.

This testing method provides the measurement of the spreading of the sebum spots (surface area) in real time directly from in vivo human skin before and after treatment. Pore size was measured with Visiopore PP39N at baseline (D0) and after 21 days of treatment (D21).

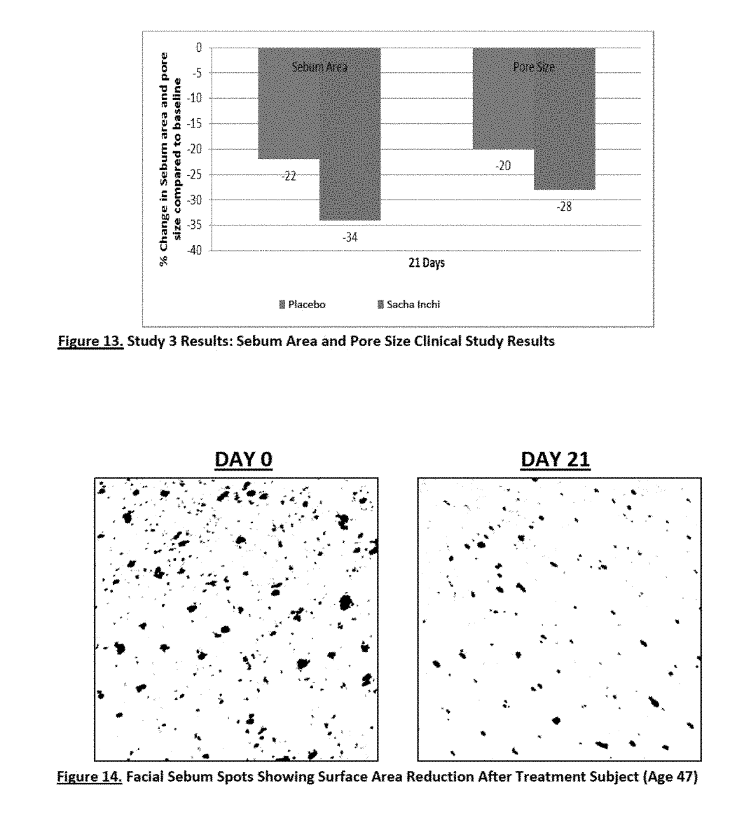

The results are plotted in FIG. 13. Shown in FIG. 13 is a bar graph indicating the results between the placebo cream and tested cream preparation for Study 3. The results indicate that it is clinically proven that topical composition containing water based extract of Sacha Inchi (MG1-31A formulation) reduced sebum area by 34% compared to baseline and performed better compared to placebo formulation after 21 days of treatment. The results represent the average of all subjects.

Furthermore, the results indicated that it is clinically proven that topical composition containing water based extract of Sacha Inchi (MG1-31A formulation) reduced the pore size by 28% compared to baseline and showed better results versus placebo after 21 days of treatment. The results represent the average of all subjects.

FIG. 14 illustrates Facial Sebum Spots Images Showing Reduction of Sebum Surface Area after 21 days of treatment from a subject of 47 years old. This representative subject showed 67% reduction in facial sebum surface area compared to baseline after 21 days of treatment.

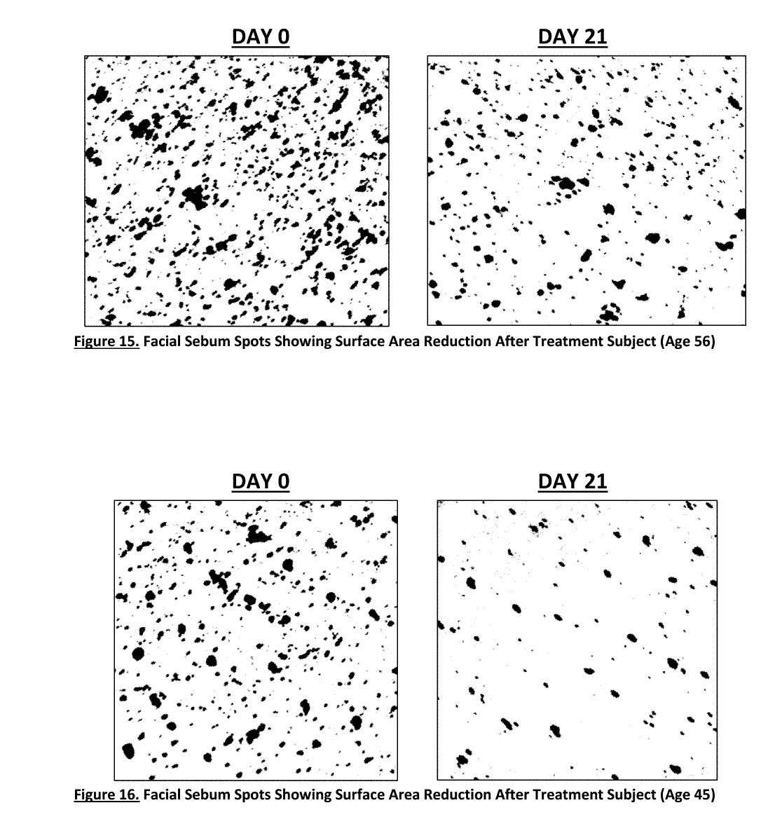

FIG. 15 illustrates Facial Sebum Spots Images Showing Reduction of Sebum Surface Area after 21 days of treatment from another subject of 56 years old. This representative subject showed 58% reduction in facial sebum surface area compared to baseline after 21 days of treatment.

FIG. 16 illustrates Facial Sebum Spots Images Showing Reduction of Sebum Surface Area after 21 days of treatment from another subject of 45 years old. This representative subject showed 39% reduction in facial sebum surface area compared to baseline after 21 days of treatment.

Depending on the embodiment, the above formulation in Study 3 may be utilized in a clay mask format. As the above results indicate, the clay mask composition with water based extract of Sacha Inchi formulation can reduce sebum production on face and be used as sebum regulator sebum reducing type mask, pore size minimizer, mattifier and smoother formulation to mask and reduce oily skin and even out skin texture and roughness.

The clay mask composition containing water based extract of Sacha Inchi was also shown to reduce acne scars, blemishes and inflammation caused by acne condition and minimizes the pore size, reduces sebum production compared to baseline data. The formulation creates a matter, smoother effect on the skin, and even texture on facial skin with daily application. This clay mask composition containing water based extract of Sacha Inchi can be used as sebum regulator, pore size minimizer and mattifier to mask the appearance of oily/greasy facial skin and smooths the rougher skin texture.

Under the experimental conditions of these clinical studies and at the formulations evaluated, these results suggest that the present invention overcomes the drawbacks of current methods to address severe skin conditions as described. The compounds tested had a positive effect on skin sagginess, firmness, skin dermis density, fine lines and deep wrinkles, roughness, hydration and moisturization of dry skin, sebum production (area), pore size and reducing oily skin condition, mattifying the skin and even out skin texture and smoothing rougher skin on face and body.

While there has been shown and described various embodiments of the instant invention it is to be appreciated that the invention may be embodied otherwise than is herein specifically shown and described and that, within said embodiments, certain changes may be made in the form and arrangement of the parts without departing from the underlying ideas or principles of this invention as set forth in the claims appended herewith.

What is claimed is:

1. A method for non-invasive treatment of skin aging conditions, including applying to a skin a topically applied composition that includes a sustainable non GMO water based extract of peptide Sacha Inchi or a hydrolyzed Plukenetia Volubilis seed extract, wherein the topically applied composition has a preparation that includes adding the sustainable non GMO water based extract of Sacha Inchi at a level range between 0.1-10% (w/w).

2. The method in claim 1, wherein the method further includes non-invasively treating for skin conditions selected from a group consisting of saggy skin, droopy skin, skin with lack of firmness and elasticity, skin with loose dermis density, skin with laxity, skin with fine lines, deep wrinkles, dry and rougher skin texture, and any combination thereof.

3. The method in claim 1, wherein the application of the topical composition containing the sustainable non GMO water based extract of peptide Sacha Inchi or the hydrolyzed Plukenetia Volubilis seed extract noninvasively provides a reduction of skin sagginess, laxity and facial volume contour including a condition of ptosis, while resculpting and reshaping a facial and a body volume contour, wherein the reduction is visibly compared to an untreated skin condition and to a placebo.

4. The method in claim 1, wherein the application of the topically applied composition visibly and non-invasively provides a decrease in a tissue gap in skin dermis, and an increase in a dermis density as compared to an untreated skin condition and a placebo to restructure, re-densify and rejuvenate skin dermis connective tissue and dermal matrix components.

5. The method of claim 1, wherein the application of the topically applied composition provides visibly less deep wrinkles and fine lines on skin as compared to an untreated skin condition and to a placebo cream.

6. The method of claim 1, wherein the topically applied composition is a revitalizing clay face firming mask, and the mask reduces sebum production on a user's face, regulates sebum, minimizes pore size, mattifies facial skin, reduces oily skin and evens out skin texture and roughness as compared to an untreated skin condition and to a placebo.

7. The method of claim 1, wherein the topically applied composition is selected from a group consisting of a day cream, an anti-aging night cream, a topical gel, a topical mask, an anti-aging day and night cream, a lotion, a gel, a mist, a spray, a toner, a serum, an anti-aging foundation, a blemish balm (BB) cream, a color corrector (CC) cream, dynamic do-all (DD) topical product, a make-up primer, a day or night moisturizer cream, a butter, an anti-aging sunscreen, an after sun formulation, a self-tanner, an anti-aging formulation, a skin lightener, a skin brightener, an anti-aging sculpting cream, a restructuring cream, a remodeling cream, a reshaping cream, a tinted topical product, and any combination thereof.

8. The method of claim 1, wherein the preparation of the topically applied composition further includes:
adding a Carbopol to water, mixing well until completely hydrated, and heating to about 70° C., then adding remaining ingredients of a Phase A;
combining a Phase B ingredients and heating to about 70 to 75° C., adding Phase B to Phase A, and mixing well;
adding a Phase C ingredients, homogenizing for 3 minutes, and switching to a propeller mixer and continue mixing to form a batch;
cooling the batch to about 40° C., and adding a Phase D ingredients one at the time and mixing until uniform;
cooling the batch to about 25° C. and adjusting for water loss;
wherein the Phase A ingredients include water, a Carbomer, a Glycerin, a Disodium EDTA; the Phase B ingredients includes a Cetearyl Alcohol and a Ceteareth 20, a Squalane, an Isopropyl Palmitate; the Phase C ingredients includes a Triethanolamine; and Phase D ingredients includes a water based extract of Sacha Inchi, Phenoxyethanol, Ethylhexylglycerin for forming an anti-sagginess, firmness, and an anti-aging facial cream composition.

9. The method of claim 8, wherein the application of the topically applied composition provides a reduction in a facial volume contour and skin sagginess while firming, reshaping and resculpting the skin, wherein the reduction is compared to an untreated skin conditions and a placebo treatment to the skin.

10. The method of claim 8, wherein the application of the topically applied composition provides a reduction in a dermis tissue gap or a non-echogenic zone, and an increase in a dermis density for restructuring and revitalizing the skin, wherein the reduction and increase is compared to an untreated skin condition and a placebo treatment to the skin.

11. The method of claim 1, wherein the preparation of the topically applied composition further includes:
adding a Carbopol to the water, mixing well until completely hydrated, and
heating to about 70° C. and adding remaining ingredients of a Phase A;
combining a Phase B ingredients in a separate vessel and heating to about 70-75° C., and adding the Phase B ingredients to the Phase A ingredients and mixing well until uniform;
adding a Phase C ingredients at about 70° C. and homogenizing for 3 minutes, then switching to a propeller mixer and continue mixing;
cooling the batch to about 40° C., and adding a Phase D ingredients one at the time and mix until uniform;
cooling the batch to about 25° C. and adjusting for water loss; and
wherein, the Phase A ingredients include Water, a Carbomer, a Glycerin, and a Disodium EDTA; and the Phase B ingredients include a Cetearyl Alcohol and a Ceteareth 20, a Caprylic Capric Triglyceride, an Isopropyl Palmitate, and an Isodecyl Neopentanoate; and the Phase C ingredients include a Triethanolamine; and the Phase D ingredients include a water based extract of Sacha Inchi and a Phenoxyethanol, an Ethylhexylglycerin, for forming an anti-wrinkle and an anti-aging cream composition.

12. The method of claim 11, wherein the application of the topically applied composition provides a reduction of a maximum depth of deepest wrinkle and wrinkle volume and a skin roughness parameters that includes an average roughness, a maximum height and an average wrinkle depth for smoothing the skin, wherein the reduction is compared to an untreated skin condition and a placebo cream applied to the skin.

13. The method of claim 11, wherein the application of the topically applied composition on the skin provides a reduction in sebum area and an oily skin condition while mattifying skin texture and skin appearance, wherein the reduction is compared to an untreated skin condition and a placebo applied to the skin.

14. The method of claim 11, wherein the topically applied composition is selected from a group consisting of a clay mask, a mud mask, a charcoal mask, a peeling a mask, a firming mask, a clarifying mask, and any combination thereof, and the topically applied composition provides a reduction of a sebum production, an oily skin, and a pore size on the skin while regulating sebum, mattifying facial skin, and evening out a skin texture and a roughness of the skin, wherein the reduction is compared to an untreated skin condition and a placebo applied to the skin.

15. The method of claim 11, wherein the topically applied composition is a clay face firming mask, and wherein the clay mask reduces acne scars, blemishes and inflammation caused by acne condition and minimizes pore size, reduces sebum production as compared to a skin baseline untreated condition, and wherein the clay mask further creates a matter, a smoother and an even texture on the skin with a daily application of the topically applied composition as compared to an untreated skin condition and a placebo applied to the skin.

16. The method of claim 1, wherein the application of the topically applied composition minimizes a pore size while smoothing, evening out a skin texture appearance and smoothing s a skin roughness as compared to a baseline untreated skin condition.

17. The method of claim 1, further includes upregulating a connective tissue growth factor gene (CTGF) and a human longevity master switch gene that includes Forkhead Box 03 (FOXO3) to provide an anti-aging benefit on the skin, wherein the upregulating is compared to an untreated skin condition and a placebo applied to the skin.

18. A composition for non-invasive treatment of skin aging conditions, comprising
a topically applied composition that includes a sustainable non GMO and a water based extract of peptide Sacha Inchi or a hydrolyzed Plukenetia Volubilis seed extract at a level between 0.1-10% solution (w/w).

19. The composition in claim 18, wherein the topically applied composition is the water based extract of Sacha Inchi or the hydrolyzed Plukenetia Volubilis seed extract having a chemical composition that includes an amino acid and a small peptide defined by having a low average molecular weight compared to an average molecular weight of a similar based extract of Sacha Inchi.

20. The composition in claim 18, wherein the topically applied composition is made with a botanical source of a Peruvian seed; and
the topically applied composition is an anti-aging facial or body topical composition to reduce saggy skin and skin laxity, improve skin firmness, increase dermal density, reshape and restructure facial and body contour and diminish fine line and deep wrinkles and smooth skin texture.

21. The composition in claim 18, wherein the low average molecular weight is less than or equal to about 2,500 Da.
